(19) United States
(12) Patent Application Publication
(10) Pub. No.: US 2021/0290791 A1
(43) Pub. Date: Sep. 23, 2021

(54) Ultraviolet Disinfection System and Method

(71) Applicant: THMGRP, Escondido, CA (US)

(72) Inventor: Marko Mandaric, Escondido, CA (US)

(21) Appl. No.: 16/822,927

(22) Filed: Mar. 18, 2020

(51) Int. Cl. A61L 2/10; A61L 2/24; A61L 2/28

(52) U.S. Cl. CPC A61L 2/10; A61L 2/24; A61L 2202/14; A61L 2202/11; A61L 2/28

(57) ABSTRACT

Apparatus and associated methods relate to measuring an ultraviolet lamp distance from a surface, determining, based on the distance, lamp illumination parameters effective to disinfect a surface area portion, and automatically disinfecting the surface based on governing the lamp illumination parameters. In an illustrative example, the distance may be measured using a passive infrared proximity sensor. The lamp may be an LED emitting a light wavelength between 180 nm and 300 nm, permitting effective disinfection. The lamp illumination parameters may include time and energy determined as functions of distance and the surface area portion exposed to illumination, permitting disinfection effectiveness adjustment as the lamp is moved. Various examples may include a counterbalancing weight directing the lamp down toward a surface, permitting efficient illumination usage. Some embodiments may advantageously include a lamp configured in a wearable bracelet or a sticker applicable to a portable device, permitting improved user access to disinfection.

### TECHNICAL FIELD

Various embodiments relate generally to disinfection.

### BACKGROUND

Disinfection is infectious microorganism destruction. Infectious microorganisms include bacterium and virus material. Some infectious microorganisms may cause infection in humans or other animals. Infection is a leading cause of medical disease. Some microorganism infections may cause severe illness, and death. An infectious illness may be a result of a microorganism invading a human or animal body. In some scenarios, a microorganism infection may use an infected human or animal body as a host to reproduce the microorganism. The production of toxins by a microorganism using a living body as a host may cause severe illness and death to the host. Destroying such microorganisms before they are able to enter a human or animal body is an object of disinfection.

Users of disinfection include individuals, public facilities, and medical service providers. Users may employ disinfection to reduce the chance of infection from microorganisms. Some users may disinfect an object before touching the object. For example, a user may disinfect an object to destroy microorganisms that may have been deposited on the object when the object was previously handled. Disinfection may destroy the structure of infectious microorganism material, rendering the infectious microorganism unable to infect, reproduce, or cause illness. Disinfection methods include the use of chemical, thermal, physical, or electromagnetic techniques to disrupt or destroy infectious microorganisms.

Access to disinfection may present challenges to a user in some scenarios. For example, a user desiring to employ a thermal disinfection technique may require an enclosure configured to heat an object to an effective temperature. A chemical disinfection technique may require transport and use of potentially hazardous chemical substances for disinfecting an object. In an illustrative example, a user of chemical disinfection agents may need protective gloves and safety eyewear, to reduce the chance of injury from handling chemical disinfection agents. In some scenarios, disinfection may be inconvenient or unavailable, and a user may expend substantial effort or resources to disinfect an object.

### SUMMARY

Apparatus and associated methods relate to measuring an ultraviolet lamp distance from a surface, determining, based on the distance, lamp illumination parameters effective to disinfect a surface area portion, and automatically disinfecting the surface based on governing the lamp illumination parameters. In an illustrative example, the distance may be measured using a passive infrared proximity sensor. The lamp may be an LED emitting a light wavelength between 180 nm and 300 nm, permitting effective disinfection. The lamp illumination parameters may include time and energy determined as functions of distance and the surface area portion exposed to illumination, permitting disinfection effectiveness adjustment as the lamp is moved. Various examples may include a counterbalancing weight directing the lamp down toward a surface, permitting efficient illumination usage. Some embodiments may advantageously include a lamp configured in a wearable bracelet or a sticker applicable to a portable device, permitting improved user access to disinfection.

Various embodiments may achieve one or more advantages. For example, some embodiments may improve a user's ease of access to disinfection. This facilitation may be a result of reducing the user's effort required to disinfect a surface, based on eliminating the need to transport and dispense chemical disinfectant material in the user's daily activities. In some embodiments, an object surface may be automatically disinfected using ultraviolet light when a user's hand passes over the object. Such automatic disinfection using ultraviolet light may reduce a user's exposure to harmful or irritating chemical disinfectants. Some embodiments may reduce the time related to a user's disinfection of objects or surfaces with ultraviolet light. Such reduced time disinfecting an object surface with ultraviolet light may be a result of adapting the ultraviolet light illumination based on the ultraviolet light distance from the surface, to improve disinfection efficiency. For example, an ultraviolet light disinfector dynamically adapting the ultraviolet light illumination may adjust disinfection effectiveness more quickly, improving the accuracy or usefulness of disinfection using ultraviolet light. Some implementations may improve a user's confidence that a surface has been effectively disinfected, based on determining the illumination energy delivered to the surface area as a function of time. Such improved user confidence in the effectiveness of disinfection may improve the user's disinfection experience.

In some embodiments, the effort required by a user to direct the ultraviolet light to the object the user desires to disinfect may be reduced. This facilitation may be a result of configuring an ultraviolet light disinfector with a counterbalancing weight adapted to direct the ultraviolet light down toward a surface to be disinfected. Some embodiments may reduce a user's effort transporting disinfection apparatus. Such reduced disinfection apparatus transportation effort may be a result of configuring an ultraviolet light disinfection apparatus in a bracelet consisting of a wearable flexible printed circuit strip looped around the user's wrist, permitting the user convenient access to the ready-to-use disinfection apparatus. For example, an ultraviolet disinfector bracelet user could automatically disinfect a surface by passing their hand over the surface, exposing a portion of the surface to disinfecting ultraviolet light. Various examples may advantageously adapt a mobile device into an ultraviolet disinfector, based on configuring an ultraviolet light disinfection apparatus in a sticker that can be applied to the mobile device. In some designs, the ultraviolet light disinfecting apparatus may employ a mobile device as a user interface, advantageously presenting the user with visually updated disinfection status for the area being disinfected.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary ultraviolet disinfector measuring an ultraviolet lamp distance from a surface, determining, based on the distance, lamp illumination parameters effective to disinfect a surface area portion, and automatically disinfecting the surface based on governing the lamp illumination parameters.

FIG. 2 depicts a schematic view of an exemplary network configured with an ultraviolet disinfector to measure an ultraviolet lamp distance from a surface, determine, based on the distance, lamp illumination parameters effective to disinfect a surface area portion, and automatically disinfect the surface based on governing the lamp illumination parameters.

FIG. 3 depicts a structural view of an exemplary ultraviolet disinfector computing device configured with an Ultraviolet Disinfection Engine (UVDE) adapted to measure an ultraviolet lamp distance from a surface, determine, based on the distance, lamp illumination parameters effective to disinfect a surface area portion, and automatically disinfect the surface based on governing the lamp illumination parameters.

FIG. 4A-4E together depict various views of exemplary ultraviolet disinfector components.

FIG. 5 depicts a process flow of an exemplary Ultraviolet Disinfection Engine (UVDE) measuring an ultraviolet lamp distance from a surface, determining, based on the distance, lamp illumination parameters effective to disinfect a surface area portion, and automatically disinfecting the surface based on governing the lamp illumination parameters.

Like reference symbols in the various drawings indicate like elements.

### DETAILED DESCRIPTION OF ILLUSTRATIVE EMBODIMENTS

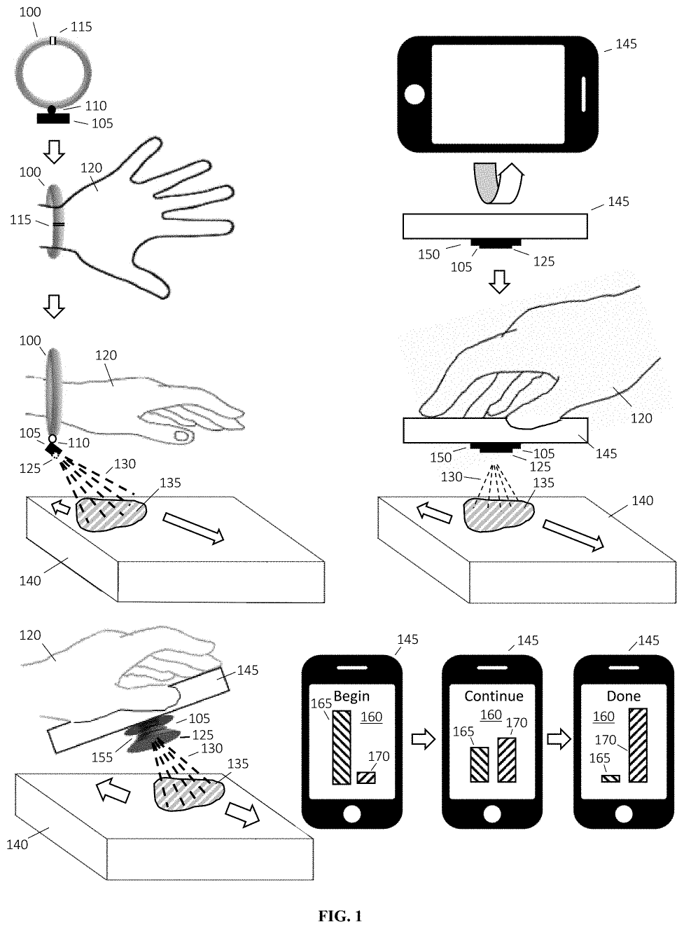

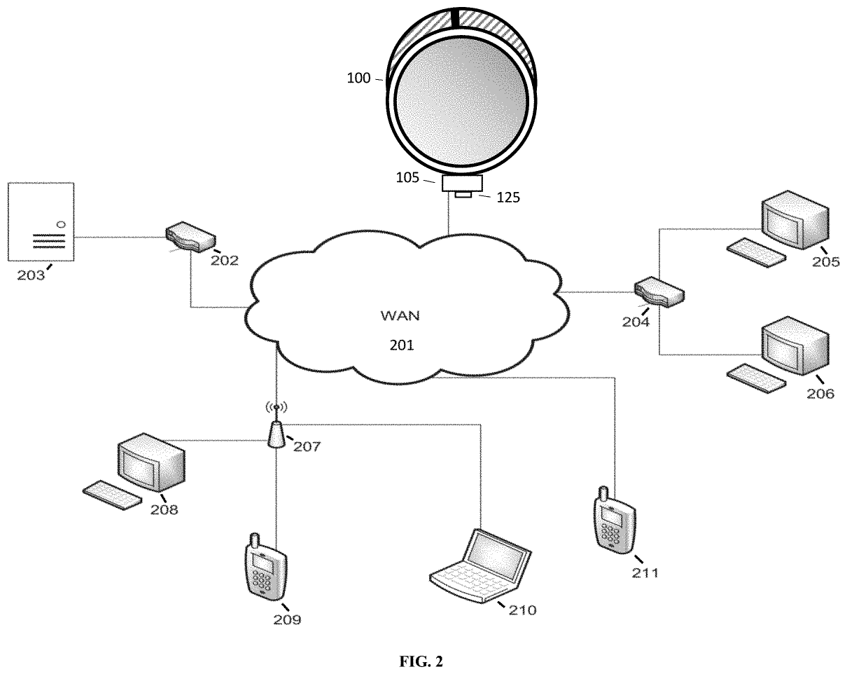

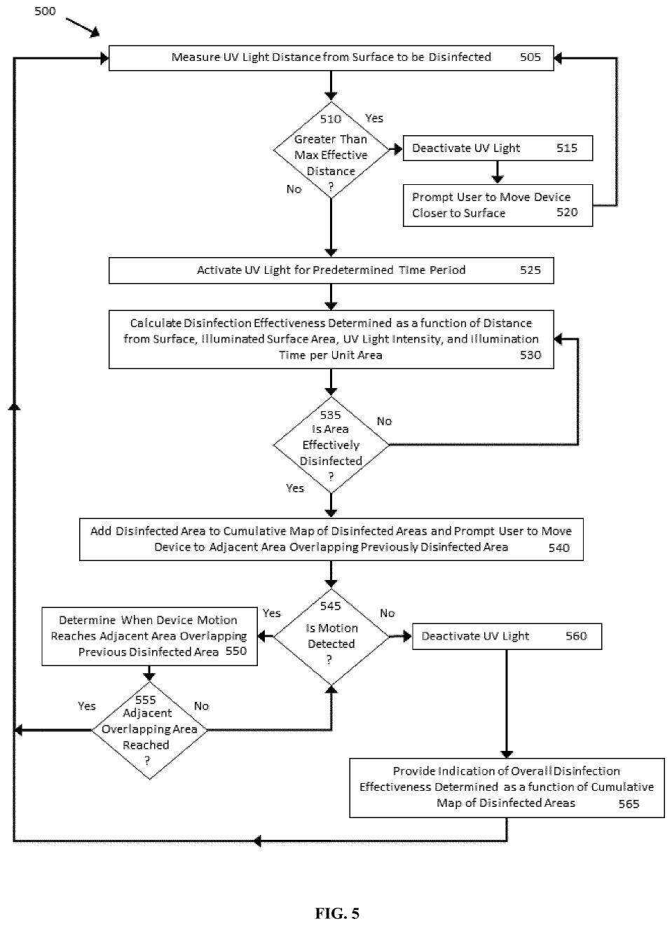

To aid understanding, this document is organized as follows. First, automatic surface disinfection with ultraviolet light adapted based on measuring the light distance from the surface is briefly introduced with reference to FIG. 1. Then, with reference to FIGS. 2-4, the discussion turns to illustrative embodiments exemplary of ultraviolet disinfector system and component design. Specifically, ultraviolet disinfector network, computing device, and structural components are disclosed. Finally, with reference to FIG. 5, an exemplary ultraviolet disinfector process flow is presented to explain improvements in ultraviolet disinfection technology.

FIG. 1 depicts an exemplary ultraviolet disinfector measuring an ultraviolet lamp distance from a surface, determining, based on the distance, lamp illumination parameters effective to disinfect a surface area portion, and automatically disinfecting the surface based on governing the lamp illumination parameters. In FIG. 1, the disinfector bracelet 100 includes the ultraviolet disinfector 105 configured with the counterbalance weight 110. In the depicted example, the disinfector bracelet 100 includes a flexible printed circuit strip configured with a clasp 115 at each strip end to secure the disinfector bracelet 100 to the user hand 120 when the strip is looped around the wrist of the hand 120. In another embodiment, the ultraviolet disinfector 105 may include one or more rigid printed circuit each configured with one or more ultraviolet disinfector 105 element connected to implement the ultraviolet disinfector 105 features. In an illustrative example, the ultraviolet disinfector 105 may be implemented with one or more interconnected printed circuit assembly configured with ultraviolet disinfector 105 components. For example, ultraviolet disinfector 105 components may include a battery, power button, proximity sensor, and an ultraviolet LED. In an illustrative example, each of the battery, power button, proximity sensor, and the ultraviolet LED may be configured in a flexible printed circuit assembly, or a rigid printed circuit assembly. Each of the battery, power button, proximity sensor, and the ultraviolet LED may be configured in one or more interconnected flexible or rigid printed circuit assembly. The one or more interconnected flexible or rigid printed circuit assembly may be operably coupled with cables connecting the battery, power button, proximity sensor, and ultraviolet LED circuit assemblies, permitting adaptation of the ultraviolet disinfector 105 physical structure to various environmental or usability requirements.

In another embodiment, the ultraviolet disinfector 105 may be included in a respirator mask. In an exemplary respirator mask ultraviolet disinfector 105 embodiment, the ultraviolet LED may be mounted to a preexisting respirator mask configured with filter holes. In an illustrative example, an ultraviolet disinfector 105 included in a respirator mask may activate the ultraviolet LED based on a pressure-activated mechanical switch driven by the respirator wearer breathing in through the mask.

In another embodiment, the ultraviolet disinfector 105 may be included in an autonomous floor-cleaning robot. In an exemplary autonomous floor-cleaning robot ultraviolet disinfector 105 embodiment, the ultraviolet LED may be mounted to the floor-cleaning robot at a distance from the floor selected to facilitate effective floor disinfection. In an illustrative example, the height of the ultraviolet LED may be adjustable by the ultraviolet disinfector 105, to achieve effective floor disinfection.

In the example illustrated by FIG. 1, the ultraviolet disinfector 105 includes the ultraviolet lamp 125 configured to emit ultraviolet radiation 130 to disinfect the target surface region 135 of the target object 140. The ultraviolet lamp 125 may be an ultraviolet LED. The ultraviolet LED may emit when activated ultraviolet light having a wavelength in the range between 180 nm and 300 nm. The ultraviolet LED may emit when activated ultraviolet light having a wavelength in the range of 222 nm and lower. In an illustrative example, the ultraviolet light wavelength may be 222 nm. In the depicted example, the counterbalance weight 110 configures the ultraviolet lamp 125 to be always pointed down toward the target surface region 135. For example, counterbalancing the weight of the ultraviolet disinfector 105 with the heavier counterbalance weight 110 directs the ultraviolet lamp 125 down toward the target surface region 135, thereby improving user safety and improving disinfection effectiveness. Directing the ultraviolet lamp 125 down toward the target surface region 135 improves safety based on avoiding misdirected ultraviolet radiation. Directing the ultraviolet lamp 125 down toward the target surface region 135 improves disinfection efficiency based on directing ultraviolet energy to the target surface region 135. The depicted ultraviolet disinfector 105 determines the distance from the ultraviolet lamp 125 to the target surface region 135. The ultraviolet disinfector 105 adjusts the ultraviolet lamp 125 illumination parameters to achieve effective disinfection of the target surface region 135 based on determining the ultraviolet radiation energy required for effective disinfection of the target surface region 135. The ultraviolet radiation energy required for effective disinfection of the target surface region 135 may be determined by the ultraviolet disinfector 105 as a function of the distance and the target surface region 135 area. In an embodiment, the ultraviolet radiation energy and ultraviolet lamp 125 illumination parameters required for effective disinfection of the target surface region 135 may be determined by a processor executing program instructions, to govern the ultraviolet lamp 125 illumination parameters determined based on sensor data. In another embodiment, the ultraviolet radiation energy and ultraviolet lamp 125 illumination parameters required for effective disinfection of the target surface region 135 may be determined by an electronic control circuit implemented without a processor, to govern the ultraviolet lamp 125 illumination parameters determined based on sensor data. The depicted ultraviolet disinfector 105 includes a proximity sensor to determine the distance to the target surface region 135. In an illustrative example, the ultraviolet lamp 125 illumination parameters governed by the ultraviolet disinfector 105 may include illumination time, light wavelength, or light energy, determined to achieve effective disinfection as a function of desired disinfection time, or as a function of available power. The ultraviolet disinfector 105 may determine the distance to the target surface region 135 based on the proximity sensor, such that the ultraviolet disinfector 105 would only activate the ultraviolet lamp 125 within a distance range to the target surface region 135 that is small enough to ensure saturation of a given area, for example, a square centimeter, to the point of high efficiency in killing off microbes. The ultraviolet lamp 125 illumination parameters required to achieve effective disinfection may be determined by the ultraviolet disinfector 105 as a function of time, the surface area to be disinfected, and the distance from the ultraviolet lamp 125 to the surface. For example, given the cumulative intensity exposure (intensity×time) in units of millijoules/cm² required to achieve disinfection of a specific microorganism contamination, the ultraviolet disinfector 105 may determine the ultraviolet lamp 125 power level and illumination time required to achieve effective disinfection based on the relationship mW/cm²×seconds=mJ/cm². The surface area to be disinfected (in cm²) is the area of the target surface region 135 in the field of view of the ultraviolet lamp 125. The area of the target surface region 135 in the field of view of the ultraviolet lamp 125 varies as a function of distance from the target surface region 135, and other factors. The ultraviolet disinfector 105 may adapt the ultraviolet lamp 125 illumination parameters based on the area of the target surface region 135 and the field of view of the ultraviolet lamp 125, to achieve effective disinfection of a given area in a desired period of time, or at a desired power level. In the depicted example, the ultraviolet disinfector 105 adapts the ultraviolet lamp 125 illumination parameters to optimize disinfection of the target surface region 135, as the user hand 120 moves back and forth across the target object 140. The ultraviolet disinfector 105 may include a motion detector, such as, for example, an accelerometer, configured to detect the user hand 120 moving back and forth across the target object 140. The ultraviolet disinfector 105 may provide a user detectable indication, such as audio or haptic feedback to the user, when the ultraviolet disinfector 105 determines the target surface region 135 has been effectively disinfected, based on evaluation of the ultraviolet lamp 125 illumination parameters as a function of time, surface area, and distance. The ultraviolet disinfector 105 may prompt the user to move the ultraviolet disinfector 105 to an adjacent region overlapping a previously disinfected surface region, to continue disinfection of the target object 140. The ultraviolet disinfector 105 may maintain a digital cumulative map of target object 140 surface regions that have been disinfected, and prompt the user to continue disinfecting surface regions that have not been adequately disinfected.

In another depicted embodiment, the mobile device 145 retains the sticker 150 including the ultraviolet disinfector 105 in an exemplary decal configuration, permitting the ultraviolet disinfector 105 to adapt the ultraviolet lamp 125 illumination parameters to optimize disinfection of the target surface region 135, as the user hand 120 moves the mobile device 145 back and forth across the target object 140.

In another depicted embodiment, the mobile device 145 grip 155 retains the ultraviolet disinfector 105 in an exemplary mobile device 145 accessory configuration, permitting the ultraviolet disinfector 105 to adapt the ultraviolet lamp 125 illumination parameters to optimize disinfection of the target surface region 135, as the user hand 120 moves the mobile device 145 back and forth across the target object 140.

In another depicted embodiment, the mobile app 160 operable via Bluetooth or other wireless connection from the mobile device 145 provides a user interface to the ultraviolet disinfector 105. In the depicted embodiment, the mobile app 160 presents visual disinfection status indications to the user. In the illustrated example, the mobile app 160 presents status indications illustrating the percentage of the target object 140 to be disinfected 165, and the percentage of the target object 140 disinfected 170. The mobile app 160 may present a visualization cumulative of the disinfected area of the target object 140 displayed as an overlay to a visualization of the target surface object 140. The visualization cumulative of the disinfected area may be determined by the ultraviolet disinfector based on the digital cumulative map of target object 140 surface regions that have been disinfected. The mobile app 160 may permit the user to select a disinfection power level, disinfection time, or disinfection effectiveness, that is, a predetermined percentage of microorganisms killed.

In an illustrative example, the mobile app 160 may provide feedback to user for pacing the disinfection procedure. For example, the mobile app 160 may direct the user concerning how quickly to move the ultraviolet disinfector 105 across the surface to achieve adequate disinfection, based on distance and movement rate. For example, the ultraviolet disinfector 105 may activate the ultraviolet lamp 125 only within a predetermined distance, such as two to four inches from a target surface. The ultraviolet disinfector 105 may turn the ultraviolet lamp 125 illumination on and off based on the distance from the surface. For example, a user may scan the ultraviolet disinfector 105 over a restaurant table, and the ultraviolet disinfector 105 may activate the ultraviolet lamp 125 when within two to four inches of the table surface. In an illustrative example, when the user moves the ultraviolet disinfector 105 up to six inches from the table, the ultraviolet disinfector 105 deactivates the ultraviolet lamp 125 and alerts the user the ultraviolet lamp 125 is deactivated. When the user moves the ultraviolet disinfector 105 back to within a predetermined effective disinfection distance from the table surface, the ultraviolet disinfector 105 reactivates the ultraviolet lamp 125. When the user moves the ultraviolet disinfector 105 past the table edge, the distance detected by the proximity sensor may be the distance to the floor, and the ultraviolet disinfector 105 may deactivate the ultraviolet lamp 125 until the user continues disinfecting by moving the ultraviolet disinfector 105 back over the table, when the ultraviolet disinfector 105 may reactivate the ultraviolet lamp 125.

FIG. 2 depicts a schematic view of an exemplary network configured with an ultraviolet disinfector to measure an ultraviolet lamp distance from a surface, determine, based on the distance, lamp illumination parameters effective to disinfect a surface area portion, and automatically disinfect the surface based on governing the lamp illumination parameters. In the example depicted by FIG. 2, the exemplary network disinfection system includes one or more application servers 203 for electronically receiving, processing and transmitting disinfection system status, configuration, or control provided by one or more ultraviolet disinfector 105 configured in one or more bracelet 100. Applications in the application server 203 may retrieve and manipulate information in storage devices and exchange information through a Network 201 (for example, the Internet, a LAN, Wi-Fi, Bluetooth, and the like). Applications in server 203 may also be used to manipulate information stored remotely and process and analyze data stored remotely across a Network 201 (for example, the Internet, a LAN, Wi-Fi, Bluetooth, and the like).

In an exemplary embodiment according to the present invention, data may be provided to the system, stored by the system and provided by the system to users of the system across local area networks (LANs) (for example, Wi-Fi networks) or wide area networks (WANs) (for example, the Internet, cellular networks). In accordance with the previous embodiment, an exemplary ultraviolet disinfector 105 system may communicate status, and receive configuration or control, to or from any number of remote computing devices (for example, servers) communicatively connected across one or more LANs and/or WANs in order to facilitate further processing of disinfection operations. One of ordinary skill in the art would appreciate that there are numerous manners in which the system could be configured and embodiments of the present invention are contemplated for use with any configuration.

According to an embodiment of the present invention, some of the applications of the present invention may not be accessible when an exemplary ultraviolet disinfector 105 system is not connected to a network, however the ultraviolet disinfector 105 systems may be able to compose disinfection results or status while offline, that will be consumed by a remote computing system when the ultraviolet disinfector 105 system is later connected to an active network.

In the exemplary network ultraviolet disinfector system depicted by FIG. 2, exchange of information through the Network 201 may occur through one or more connections. In some cases, connections may be over-the-air (OTA), passed through networked systems, directly connected to one or more Network 201 or directed through one or more router 202. The one or more router 202 is completely optional and other embodiments in accordance with the present invention may or may not utilize one or more router 202. One of ordinary skill in the art would appreciate that there are numerous ways server 203 may connect to Network 201 for the exchange of information with an ultraviolet disinfector 105 system or other devices (for example, end user computing devices), and embodiments of the present invention are contemplated for use with any method for connecting to networks for the purpose of exchanging information. Further, while this application refers to high speed connections, embodiments of the present invention may be utilized with connections of any speed.

In an embodiment of the present invention, an exemplary ultraviolet disinfector 105 may connect to server 203 via Network 201. The server 203, upon receiving and processing disinfection status or indication from the ultraviolet disinfector 105, may provide processed disinfection status or indication information to end users of the system, such as: i) through feedback directly to a mobile app associated with the ultraviolet disinfector 105, such mobile app being directly connected to the Network 201, with processed disinfection status or indication information being provided through one or more processing means associated with the mobile app (for example, mobile device), ii) through a computing device 205, 206 connected to the WAN 201 through a routing device 204, iii) through a computing device 208, 209, 210 connected to a wireless access point 207 or iv) through a computing device 211 via a wireless connection (e.g., CDMA, GMS, 3G, 4G) to the Network 201. One of ordinary skill in the art would appreciate that there are numerous ways that a component may connect to server 203 via Network 201, and embodiments of the present invention are contemplated for use with any method for connecting to server 203 via Network 201. Furthermore, server 203 could be comprised of a personal computing device, such as a smartphone, acting as a host for other computing devices to connect to.

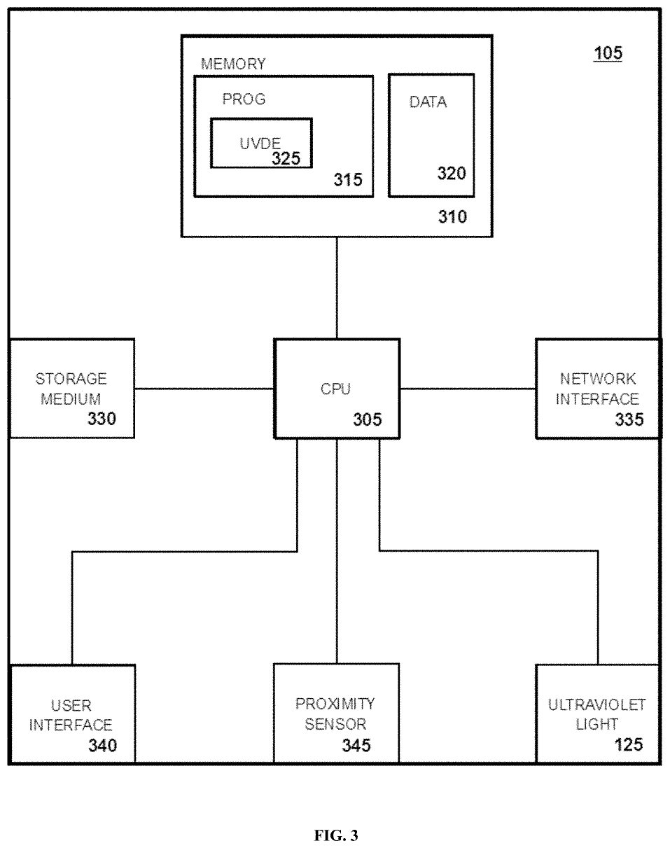

FIG. 3 depicts a structural view of an exemplary ultraviolet disinfector computing device configured with an Ultraviolet Disinfection Engine (UVDE) adapted to measure an ultraviolet lamp distance from a surface, determine, based on the distance, lamp illumination parameters effective to disinfect a surface area portion, and automatically disinfect the surface based on governing the lamp illumination parameters. In FIG. 3, the block diagram of the exemplary ultraviolet disinfector 105 includes the processor 305 and the memory 310. The processor 305 is in electrical communication with the memory 310. The depicted memory 310 includes the program memory 315 and the data memory 320. The depicted program memory 315 includes processor-executable program instructions implementing the UVDE (Ultraviolet Disinfection Engine) 325. The illustrated program memory 315 may include processor-executable program instructions configured to implement an OS (Operating System). The OS may include processor executable program instructions configured to implement various operations when executed by the processor 305. The OS may be omitted. The illustrated program memory 315 may include processor-executable program instructions configured to implement various Application Software. The Application Software may include processor executable program instructions configured to implement various operations when executed by the processor 305. The Application Software may be omitted. In the depicted embodiment, the processor 305 is communicatively and operably coupled with the storage medium 330. In the depicted embodiment, the processor 305 is communicatively and operably coupled with the Network Interface 335. The network interface may be a wireless network interface. The network interface may be a Wi-Fi interface. The network interface may be a Bluetooth interface. In an illustrative example, the ultraviolet disinfector 105 may include more than one network interface. The network interface may be a wireline interface. The network interface may be omitted. In the depicted embodiment, the processor 305 is communicatively and operably coupled with the user interface 340. The user interface 340 may be adapted to receive input from a user or send output to a user. The user interface 340 may be adapted to an input-only or output-only user interface mode. The user interface 340 may include an imaging display. The user interface 340 may include an audio interface. The audio interface may include an audio input. The audio interface may include an audio output. The user interface 340 may be touch-sensitive. The user interface 340 may be implemented by a mobile app configured in a device external to the ultraviolet disinfector 105. The user interface 340 may include user operable controls such as, for example, a power switch, and a control which when activated initiates disinfection operations. The user interface 340 may include user detectable indicators, such as, for example, an audio tone or beep generator, a vibration device to provide haptic feedback to the user, and the like. In the depicted embodiment, the ultraviolet disinfector 105 includes an accelerometer operably coupled with the processor 305. The ultraviolet disinfector 105 may include a GPS module operably coupled with the processor 305. The ultraviolet disinfector 105 may include a magnetometer operably coupled with the processor 305. The ultraviolet disinfector 105 may include a power supply configured to operably power the ultraviolet disinfector 105. The power supply may be a battery. In the depicted embodiment, the processor 305 is communicatively and operably coupled with the proximity sensor 345. The proximity sensor 345 may be a passive infrared (IR) proximity sensor. The proximity sensor 345 may be an ultrasonic proximity sensor. The proximity sensor 345 may be a ToF (Time of Flight) optical sensor. The proximity sensor 345 may be a capacitive proximity sensor. In the depicted embodiment, the processor 305 is communicatively and operably coupled with the ultraviolet (UV) light 125. The UV light 125 may be a UV LED. Useful examples of the illustrated ultraviolet disinfector 105 include, but are not limited to, personal computers, servers, tablet PCs, smartphones, or other computing devices. Multiple ultraviolet disinfector 105 devices may be operably linked to form a computer network in a manner as to distribute and share one or more resources, such as clustered computing devices and server banks/farms. Examples of such general-purpose multi-unit computer networks suitable for embodiments of the disclosure, their typical configuration and many standardized communication links are well known to one skilled in the art, as explained in more detail in the foregoing FIG. 2 description. An exemplary ultraviolet disinfector 105 design may be realized in a distributed implementation. In an illustrative example, an ultraviolet disinfector 105 design may be partitioned between a client device, such as, for example, a phone, and, a more powerful server system, as depicted, for example, in FIG. 2. An ultraviolet disinfector 105 partition hosted on a PC or mobile device may choose to delegate some parts of computation, such as, for example, machine learning or deep learning, to a host server. A client device partition may delegate computation-intensive tasks to a host server to take advantage of a more powerful processor, or to offload excess work. In an illustrative example, some devices may be configured with, for example, a GPU, or a mobile chip including an engine adapted to implement specialized processing, such as, for example, neural networks, machine learning, artificial intelligence, image recognition, audio processing, or digital signal processing. For example, such an engine adapted to specialized processing may have sufficient processing power to implement some features. However, an exemplary ultraviolet disinfector 105 may be configured to operate on a device with less processing power, such as, for example, various gaming consoles, which may not have sufficient processor power, or a suitable CPU architecture, to adequately support ultraviolet disinfector 105. Designs configured to operate on a such a device with reduced processor power may work in conjunction with a more powerful server system.

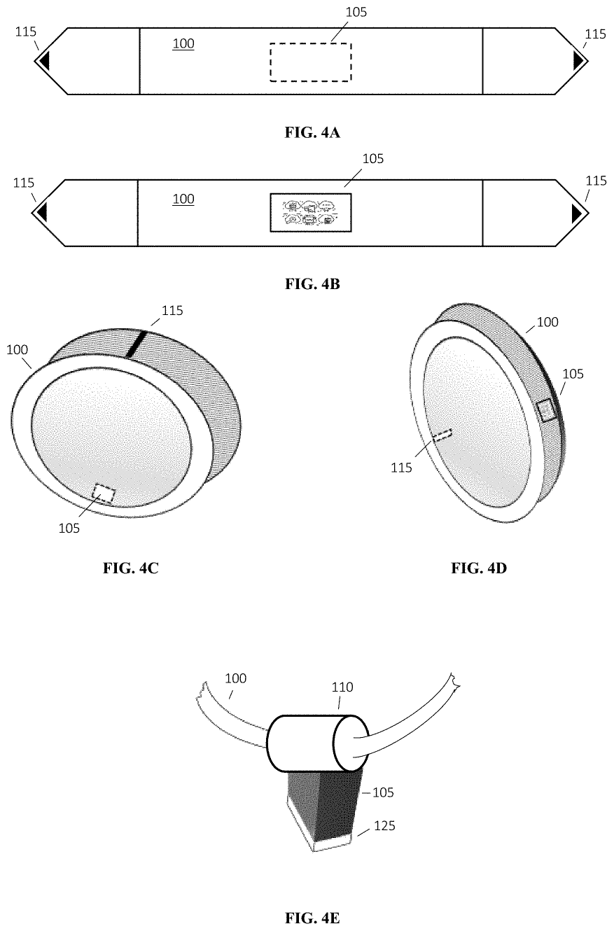

FIG. 4A-4E together depict various views of exemplary ultraviolet disinfector components. In FIG. 4A, the exemplary bracelet 100 flexible printed circuit strip inner surface is illustrated in an exemplary unlatched configuration depicting the ultraviolet disinfector 105 position on the strip, and the clasp 115 at each bracelet 100 flexible printed circuit strip end. In an illustrative example, an exemplary disinfector bracelet 100 constructed using the illustrated flexible printed circuit strip substrate as the disinfector may implement the complete device physical structure with the flexible printed circuit strip. Such a disinfector bracelet 100 design would not require a housing, reducing cost and improving device operational disinfection positioning precision based on the user's hand motion. In an illustrative example, the clasp 115 on each end of the flexible printed circuit strip may be configured to join the two flexible printed circuit strip ends together. For example, each clasp 115 may be a metal clasp 115 configured to join the two flexible printed circuit strip ends together via magnet. In an illustrative example, the counter weight of the two metal clasp 115 endpoints may be secured to the flexible printed circuit strip. The two metal clasp 115 endpoints may be glued to the flexible printed circuit strip. In FIG. 4B, the exemplary bracelet 100 flexible printed circuit strip outer surface is illustrated in an exemplary unlatched configuration depicting the ultraviolet disinfector 105 retained on the strip, and the clasp 115 at each bracelet 100 flexible printed circuit strip end. In FIG. 4C, the bracelet 100 top perspective view depicts the flexible printed circuit strip in an exemplary latched configuration, illustrating the ultraviolet disinfector 105 position from the strip inner surface. In the depicted example, the strip is joined by the clasp 115 at each bracelet 100 flexible printed circuit strip end. In FIG. 4D, the bracelet 100 bottom perspective view depicts the flexible printed circuit strip in an exemplary latched configuration, illustrating the ultraviolet disinfector 105 on the strip outer surface. In the depicted example, the strip is joined by the clasp 115 at each bracelet 100 flexible printed circuit strip end. In FIG. 4E, the counterbalance weight 110 configures the ultraviolet lamp 125 to be always pointed down toward a target surface from the ultraviolet disinfector 105. In an illustrative example, counterbalancing the weight of the ultraviolet disinfector 105 (for example, the ultraviolet disinfector 105 weight may be due in part to a battery configured to power the device) with the heavier counterbalance weight 110 directs the ultraviolet lamp 125 down toward the target surface region 135, thereby improving user safety and improving disinfection effectiveness.

FIG. 5 depicts a process flow of an exemplary Ultraviolet Disinfection Engine (UVDE) measuring an ultraviolet lamp distance from a surface, determining, based on the distance, lamp illumination parameters effective to disinfect a surface area portion, and automatically disinfecting the surface based on governing the lamp illumination parameters. The method depicted in FIG. 5 is given from the perspective of the UVDE 325 implemented via processor-executable program instructions executing on the ultraviolet disinfector 105 processor 305, depicted in FIG. 3. In the illustrated embodiment, the UVDE 325 executes as program instructions on the processor 305 configured in the UVDE 325 host ultraviolet disinfector 105, depicted in at least FIG. 1, FIG. 2, FIG. 3, and FIG. 4. The UVDE 325 may execute as a cloud service communicatively and operatively coupled with system services, hardware resources, or software elements local to and/or external to the UVDE 325 host ultraviolet disinfector 105. The depicted method 500 begins at step 505 with the processor 305 measuring the UV light distance from a surface to be disinfected. The method 500 may be invoked in response to a user control action initiating the disinfection procedure.

At step 510 the processor 305 performs a test to determine if the distance measured by the processor 305 at step 505 is greater than a predetermined maximum effective disinfection distance. The maximum effective disinfection distance may be predetermined as a function of UV energy required to kill a predetermined percentage of a particular microorganism type. The microorganism type and the percentage of the microorganism to kill may be selected by a user. The microorganism type and the percentage of the microorganism to kill may be received from a cloud server coordinating the disinfection performance of a group of ultraviolet disinfector 105 devices configured according to a disinfection policy. In an illustrative example, the disinfection policy may be a policy implemented enterprise-wide by an organization such as a hospital, or for persons afflicted with a common immune deficiency rendering those persons particularly susceptible to a certain microorganism type.

Upon a determination by the processor 305 at step 510 the distance measured by the processor 305 at step 505 is greater than the predetermined maximum effective disinfection distance, the method continues at step 515 with the processor 305 deactivating the UV light. The method continues a step 520 with the processor 305 prompting the user to move the device closer to the surface to be disinfected, and the method continues at step 505 with the processor 305 measuring the UV light distance from a surface to be disinfected.

Upon a determination by the processor 305 at step 510 the distance measured by the processor 305 at step 505 is not greater than the predetermined maximum effective disinfection distance, the method continues at step 525 with the processor 305 activating the UV light for a predetermined time period. The UV light activation time period may be determined as a function of the UV energy required to kill a percentage of a particular microorganism with a given UV light wavelength delivered to a surface area calculated based on the UV light field of view and distance from the surface to be disinfected.

At step 530 the processor 305 calculates the disinfection effectiveness determined as a function of the distance from the surface, the surface area illuminated by the UV light, the UV light intensity, and illumination time per unit area.

At step 535 the processor 305 performs a test to determine if the area is effectively disinfected. Upon a determination by the processor 305 at step 535 the area is not effectively disinfected, the method continues at step 530 with the processor 305 calculating the disinfection effectiveness while the UV light is active.

Upon a determination by the processor 305 at step 535 the area is effectively disinfected, the method continues at step 540 with the processor 305 adding the disinfected area to a cumulative map of disinfected areas, and prompting the user to move the device to an adjacent area overlapping the previously disinfected area.

The method continues at step 545 with the processor 305 performing a test to determine if device motion is detected. Upon a determination by the processor 305 at step 545 device motion is not detected, the method continues at step 560 with the processor deactivating the UV light, and the method continues at step 565 with the processor 305 providing an indication of overall disinfection effectiveness determined as a function of the cumulative map of disinfected areas and the disinfection effectiveness determined by the processor 305 at step 535 for each disinfected area. Then, the method continues at step 505 with the processor 305 measuring the UV light distance from a surface to be disinfected.

Upon a determination by the processor 305 at step 545 device motion is detected, the method continues at step 550 with the processor 305 monitoring the device motion to determine when the device reaches an adjacent area overlapping the previous disinfected area. Then, the method continues at step 555 with the processor 305 performing a test to determine if the device motion reached an adjacent overlapping area.

Upon a determination by the processor 305 at step 555 the device motion did not reach an adjacent overlapping area, the method continues at step 545, with the processor 305 performing a test to determine if device motion is detected. Upon a determination by the processor 305 at step 555 the device motion reached an adjacent overlapping area, the method continues at step 505 with the processor 305 measuring the UV light distance from a surface to be disinfected. In various embodiments, the method may repeat. In some designs, the method may terminate. In an example illustrative of various implementations, the method may restart with different configuration or parameters.

In the Summary above and in this Detailed Description, and the Claims below, and in the accompanying drawings, reference is made to particular features of various embodiments of the invention. It is to be understood that the disclosure of embodiments of the invention in this specification is to be interpreted as including all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In the present disclosure, various features may be described as being optional, for example, through the use of the verb “may;” or, through the use of any of the phrases: “in some embodiments,” “in an embodiment,” “in one embodiment,” “in another embodiment,” “in some implementations,” “in some designs,” “in various embodiments,” “in various implementations,” “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

In various embodiments, elements described herein as coupled or connected may have an effectual relationship realizable by a direct connection or indirectly with one or more other intervening elements.

In the present disclosure, the term “any” may be understood as designating any number of the respective elements, for example, as designating one, at least one, at least two, each or all of the respective elements. Similarly, the term “any” may be understood as designating any collection(s) of the respective elements, for example, as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

The Abstract is provided to comply with 37 C. F. R. § 1.72(b), to allow the reader to quickly ascertain the nature of the technical disclosure and is submitted with the understanding that it will not be used to interpret or limit the scope or meaning of the claims.

In the present disclosure, all embodiments where “comprising” is used may have as alternatives “consisting essentially of,” or “consisting of.” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, B and C can consist of (that is, contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described hereinabove may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f). Elements recited in means-plus-function format are intended to be construed in accordance with 35 U.S.C. § 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or references to an embodiment or embodiments, or variations thereof, as recited throughout this specification, are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects may lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

According to an embodiment of the present invention, the system and method may be accomplished through the use of one or more computing devices. As depicted, for example, at least in FIG. 2 and FIG. 3, one of ordinary skill in the art would appreciate that an exemplary system appropriate for use with embodiments in accordance with the present application may generally include one or more of a Central processing Unit (CPU), Random Access Memory (RAM), a storage medium (e.g., hard disk drive, solid state drive, flash memory, cloud storage), an operating system (OS), one or more application software, a display element, one or more communications means, or one or more input/output devices/means. Examples of computing devices usable with embodiments of the present invention include, but are not limited to, proprietary computing devices, personal computers, mobile computing devices, tablet PCs, mini-PCs, servers or any combination thereof. The term computing device may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device.

In various embodiments, communications means, data store(s), processor(s), or memory may interact with other components on the computing device, in order to effect the provisioning and display of various functionalities associated with the system and method detailed herein. One of ordinary skill in the art would appreciate that there are numerous configurations that could be utilized with embodiments of the present invention, and embodiments of the present invention are contemplated for use with any appropriate configuration.

According to an embodiment of the present invention, the communications means of the system may be, for instance, any means for communicating data over one or more networks or to one or more peripheral devices attached to the system. Appropriate communications means may include, but are not limited to, circuitry and control systems for providing wireless connections, wired connections, cellular connections, data port connections, Bluetooth connections, or any combination thereof. One of ordinary skill in the art would appreciate that there are numerous communications means that may be utilized with embodiments of the present invention, and embodiments of the present invention are contemplated for use with any communications means.

Throughout this disclosure and elsewhere, block diagrams and flowchart illustrations depict methods, apparatuses (that is, systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “circuit,” “module,” or “system.”

While the foregoing drawings and description may set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a sequence of computational instructions or program instructions. It will be appreciated that a programmable apparatus (i.e., computing device) can receive such a computer program and, by processing the computational instructions thereof, produce a further technical effect.

A programmable apparatus may include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

The embodiments disclosed hereinabove may be summarized as follows.

### Embodiment 1

An ultraviolet disinfector apparatus, comprising: an ultraviolet lamp; a processor, operably coupled with the lamp to govern the lamp illumination; a proximity sensor, operably coupled with the processor, the proximity sensor configured to send to the processor proximity sensor data encoding the lamp distance from a surface; and, a memory that is not a transitory propagating signal, the memory operably coupled with the processor and encoding computer readable instructions, including processor executable program instructions, the computer readable instructions accessible to the processor, wherein the processor executable program instructions, when executed by the processor, cause the processor to perform operations comprising: measure the ultraviolet lamp distance from the surface; determine, based on the distance, lamp illumination parameters effective to disinfect a surface portion; and, automatically disinfect the surface based on governing the lamp illumination parameters.

### Embodiment 2

The apparatus of embodiment 1, wherein the ultraviolet lamp further comprises an LED configured to emit when activated ultraviolet light.

### Embodiment 3

The apparatus of embodiment 2, wherein the ultraviolet light further comprises a wavelength between 180 nm and 300 nm.

### Embodiment 4

The apparatus of embodiment 1, wherein the ultraviolet lamp energy is adjustable by the processor.

### Embodiment 5

The apparatus of embodiment 1, wherein measure the ultraviolet lamp distance further comprises receive by the processor sensor data captured by the proximity sensor.

### Embodiment 6

The apparatus of embodiment 1, wherein the proximity sensor further comprises a passive infrared proximity sensor.

### Embodiment 7

The apparatus of embodiment 1, wherein the lamp illumination parameters further comprise illumination time.

### Embodiment 8

The apparatus of embodiment 1, wherein the lamp illumination parameters further comprise illumination energy.

### Embodiment 9

The apparatus of embodiment 1, wherein the apparatus further comprises a flexible printed circuit strip operably retaining the processor, lamp, proximity sensor, and memory, wherein the flexible printed circuit strip has two ends, and wherein each flexible printed circuit strip end includes a clasp configured to releasably secure the flexible printed circuit strip to a human wrist based on joining each end to the other end when the flexible printed circuit strip is looped to subsume the wrist.

### Embodiment 10

The apparatus of embodiment 1, wherein the apparatus further comprises a sticker operably retaining the processor, lamp, proximity sensor, and memory, wherein the sticker is configured to secure the apparatus to a portable device.

### Embodiment 11

The apparatus of embodiment 1, wherein the apparatus further comprises a counterbalancing weight configured to direct the lamp down toward a surface.

### Embodiment 12

An ultraviolet disinfector apparatus, comprising: an ultraviolet LED configured to emit when activated ultraviolet light having a wavelength between 180 nm and 300 nm; a processor, operably coupled with the ultraviolet LED to govern the ultraviolet LED illumination; a passive infrared proximity sensor, operably coupled with the processor, the passive infrared proximity sensor configured to send to the processor sensor data encoding the ultraviolet LED distance from a surface; and, a memory that is not a transitory propagating signal, the memory operably coupled with the processor and encoding computer readable instructions, including processor executable program instructions, the computer readable instructions accessible to the processor, wherein the processor executable program instructions, when executed by the processor, cause the processor to perform operations comprising: measure the ultraviolet LED distance from the surface based on receiving sensor data captured by the proximity sensor; determine, based on the ultraviolet LED distance from the surface, ultraviolet LED illumination parameters effective to disinfect a surface portion exposed to the light emitted by the LED when the LED is illuminated, wherein the ultraviolet LED illumination parameters further comprise LED illumination time determined as functions of the area of the surface portion exposed to the light and the LED illumination energy; and, automatically disinfect the surface based on governing the ultraviolet LED illumination parameters; and, a flexible printed circuit strip operably retaining the processor, ultraviolet LED, passive infrared proximity sensor, and memory, wherein the flexible printed circuit strip has two ends, and wherein each flexible printed circuit strip end includes a clasp configured to releasably secure the flexible printed circuit strip to a human wrist based on joining each end to the other end when the flexible printed circuit strip is looped to subsume the wrist; and, a counterbalancing weight configured to direct the ultraviolet LED down toward a surface.

### Embodiment 13

The apparatus of embodiment 12, wherein the apparatus further comprises an accelerometer, operably coupled with the processor, wherein the accelerometer is configured to send to the processor sensor data encoding acceleration due to the apparatus motion, and wherein the flexible printed circuit strip further retains the accelerometer.

### Embodiment 14

The apparatus of embodiment 13, wherein automatically disinfect the surface further comprises determine the time the surface portion is illuminated by the ultraviolet LED determined as a function of the surface portion area and apparatus motion determined based on accelerometer data.

### Embodiment 15

The apparatus of embodiment 14, wherein the ultraviolet LED illumination parameters further comprise ultraviolet LED illumination time determined as a function of the illumination energy delivered to the surface portion, wherein the illumination energy delivered to the surface portion is determined based on the area of the surface portion illuminated by the ultraviolet LED.

### Embodiment 16

The apparatus of embodiment 12, wherein the operations performed by the processor further comprise determine if the area of the surface portion exposed to the light has been disinfected, based on the ultraviolet LED illumination time, the ultraviolet LED illumination energy, and the area of the surface portion exposed to the light.

### Embodiment 17

The apparatus of embodiment 16, wherein the operations performed by the processor further comprise in response to determining the area of the surface portion exposed to the light has been disinfected, provide a user detectable indication the surface portion has been disinfected; and, direct the user to move the apparatus to illuminate an adjacent surface portion overlapping the disinfected surface portion using the ultraviolet LED.

### Embodiment 18

An ultraviolet disinfector apparatus, comprising: an ultraviolet LED configured to emit when activated ultraviolet light having a wavelength between 180 nm and 300 nm; a processor, operably coupled with the ultraviolet LED to govern the ultraviolet LED illumination; an accelerometer, operably coupled with the processor, wherein the accelerometer is configured to send to the processor sensor data encoding acceleration due to the apparatus motion; a passive infrared proximity sensor, operably coupled with the processor, the passive infrared proximity sensor configured to send to the processor sensor data encoding the ultraviolet LED distance from a surface; and, a memory that is not a transitory propagating signal, the memory operably coupled with the processor and encoding computer readable instructions, including processor executable program instructions, the computer readable instructions accessible to the processor, wherein the processor executable program instructions, when executed by the processor, cause the processor to perform operations comprising: measure the ultraviolet LED distance from the surface based on receiving sensor data captured by the proximity sensor; determine, based on the ultraviolet LED distance from the surface, ultraviolet LED illumination parameters effective to disinfect a surface portion exposed to the light emitted by the ultraviolet LED when the ultraviolet LED is illuminated, wherein the ultraviolet LED illumination parameters further comprise ultraviolet LED illumination time determined as functions of the area of the surface portion exposed to the light and the ultraviolet LED illumination energy; automatically disinfect the surface portion by governing the ultraviolet LED illumination parameters adjusted as a function of the area of the surface portion illuminated by the ultraviolet LED and apparatus motion determined based on accelerometer data; determine if the area of the surface portion exposed to the light has been disinfected, based on the ultraviolet LED illumination time, the ultraviolet LED illumination energy, and the area of the surface portion exposed to the light; and, in response to determining the area of the surface portion exposed to the light has been disinfected: provide a user detectable indication the surface portion has been disinfected; and, direct the user to move the apparatus to an adjacent surface portion overlapping the disinfected surface portion.

### Embodiment 19

The apparatus of embodiment 18, wherein the apparatus further comprises a flexible printed circuit strip operably retaining the processor, accelerometer, ultraviolet LED, passive infrared proximity sensor, and memory, wherein the flexible printed circuit strip has two ends, wherein each flexible printed circuit strip end includes a clasp configured to releasably secure the flexible printed circuit strip to a human wrist based on joining each end to the other end when the flexible printed circuit strip is looped to subsume the wrist, and wherein the ultraviolet LED is configured in the flexible printed circuit strip with a counterbalancing weight adapted to direct the ultraviolet LED down toward a surface.

### Embodiment 20

The apparatus of embodiment 19, wherein the apparatus further comprises a sticker operably retaining the processor, lamp, proximity sensor, and memory, wherein the sticker is configured to secure the apparatus to a portable device.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. An ultraviolet disinfector apparatus, comprising:
an ultraviolet lamp;
a control circuit, operably coupled with the lamp to govern the lamp illumination; and,
a proximity sensor, operably coupled with the control circuit, the proximity sensor configured to send to the control circuit a proximity sensor signal encoding the lamp distance from a surface, causing the control circuit to:
measure the ultraviolet lamp distance from the surface;
determine, based on the distance, lamp illumination parameters effective to disinfect a surface portion; and,
automatically disinfect the surface based on governing the lamp according to the illumination parameters.

2. The apparatus of claim 1, wherein the ultraviolet lamp further comprises an LED configured to emit when activated ultraviolet light.

3. The apparatus of claim 2, wherein the ultraviolet light further comprises a wavelength between 180 nm and 300 nm.

4. The apparatus of claim 1, wherein the ultraviolet lamp energy is adjustable by the control circuit.

5. The apparatus of claim 1, wherein measure the ultraviolet lamp distance further comprises receive by the control circuit the signal from the proximity sensor.

6. The apparatus of claim 1, wherein the proximity sensor further comprises a passive infrared proximity sensor.

7. The apparatus of claim 1, wherein the lamp illumination parameters further comprise illumination time.

8. The apparatus of claim 1, wherein the lamp illumination parameters further comprise illumination energy.

9. The apparatus of claim 1, wherein the control circuit further comprises: an operational amplifier having an output, a non-inverting input, an inverting input, a positive supply rail, and a negative supply rail; wherein the output is configured to drive the ultraviolet lamp illumination governed as a function of a control voltage applied between the non-inverting input and the control circuit ground, and wherein the control voltage is determined as a function of the proximity sensor connected between the non-inverting input, the positive supply rail, and the negative supply rail.

10. The apparatus of claim 1, wherein the control circuit further comprises:
a processor, operably coupled with the lamp to govern the lamp illumination; and,
a memory that is not a transitory propagating signal, the memory operably coupled with the processor and encoding computer readable instructions, including processor executable program instructions, the computer readable instructions accessible to the processor, wherein the processor executable program instructions, when executed by the processor, cause the processor to perform operations comprising:
measure the ultraviolet lamp distance from the surface;
determine, based on the distance, lamp illumination parameters effective to disinfect a surface portion; and,
automatically disinfect the surface based on governing the lamp according to the illumination parameters.

11. The apparatus of claim 10, wherein the apparatus further comprises a flexible printed circuit strip operably retaining the ultraviolet disinfector, wherein the flexible printed circuit strip has two ends, and wherein each flexible printed circuit strip end includes a clasp configured to releasably secure the flexible printed circuit strip to a human wrist based on joining each end to the other end when the flexible printed circuit strip is looped to subsume the wrist.

12. The apparatus of claim 1, wherein the apparatus further comprises a sticker operably retaining the ultraviolet disinfector, wherein the sticker is configured to secure the apparatus to a portable device.

13. The apparatus of claim 1, wherein the apparatus further comprises a counterbalancing weight configured to direct the lamp down toward a surface.

14. An ultraviolet disinfector apparatus, comprising:
an ultraviolet LED configured to emit when activated ultraviolet light having a wavelength between 180 nm and 300 nm;
a processor, operably coupled with the ultraviolet LED to govern the ultraviolet LED illumination;
a passive infrared proximity sensor, operably coupled with the processor, the passive infrared proximity sensor configured to send to the processor sensor data encoding the ultraviolet LED distance from a surface; and,
a memory that is not a transitory propagating signal, the memory operably coupled with the processor and encoding computer readable instructions, including processor executable program instructions, the computer readable instructions accessible to the processor, wherein the processor executable program instructions, when executed by the processor, cause the processor to perform operations comprising:
measure the ultraviolet LED distance from the surface based on receiving sensor data captured by the proximity sensor;
determine, based on the ultraviolet LED distance from the surface, ultraviolet LED illumination parameters effective to disinfect a surface portion exposed to the light emitted by the LED when the LED is illuminated, wherein the ultraviolet LED illumination parameters further comprise LED illumination time determined as functions of the area of the surface portion exposed to the light and the LED illumination energy; and,
automatically disinfect the surface based on governing the ultraviolet LED operation in accordance with the illumination parameters; and,
a flexible printed circuit strip operably retaining the processor, ultraviolet LED, passive infrared proximity sensor, and memory, wherein the flexible printed circuit strip has two ends, and wherein each flexible printed circuit strip end includes a clasp configured to releasably secure the flexible printed circuit strip to a human wrist based on joining each end to the other end when the flexible printed circuit strip is looped to subsume the wrist; and,
a counterbalancing weight configured to direct the ultraviolet LED down toward a surface.

15. The apparatus of claim 14, wherein the apparatus further comprises an accelerometer, operably coupled with the processor, wherein the accelerometer is configured to send to the processor sensor data encoding acceleration due to the apparatus motion, and wherein the flexible printed circuit strip further retains the accelerometer.

16. The apparatus of claim 15, wherein automatically disinfect the surface further comprises determine the time the surface portion is illuminated by the ultraviolet LED determined as a function of the surface portion area and apparatus motion determined based on accelerometer data.

17. The apparatus of claim 14, wherein the ultraviolet LED illumination parameters further comprise ultraviolet LED illumination energy determined as a function of the area of the surface portion illuminated by the ultraviolet LED and the LED distance from the surface.

18. The apparatus of claim 14, wherein the operations performed by the processor further comprise determine if the area of the surface portion exposed to the light has been disinfected, based on the ultraviolet LED illumination time, the ultraviolet LED illumination energy, and the area of the surface portion exposed to the light.

19. The apparatus of claim 18, wherein the operations performed by the processor further comprise in response to determining the area of the surface portion exposed to the light has been disinfected, provide a user detectable indication the surface portion has been disinfected; and, direct the user to move the apparatus to illuminate an adjacent surface portion overlapping the disinfected surface portion using the ultraviolet LED.

20. An ultraviolet disinfector apparatus, comprising:
an ultraviolet LED configured to emit when activated ultraviolet light having a wavelength between 180 nm and 300 nm;
a processor, operably coupled with the ultraviolet LED to govern the ultraviolet LED illumination;
an accelerometer, operably coupled with the processor, wherein the accelerometer is configured to send to the processor sensor data encoding acceleration due to the apparatus motion;
a passive infrared proximity sensor, operably coupled with the processor, the passive infrared proximity sensor configured to send to the processor sensor data encoding the ultraviolet LED distance from a surface; and,
a memory that is not a transitory propagating signal, the memory operably coupled with the processor and encoding computer readable instructions, including processor executable program instructions, the computer readable instructions accessible to the processor, wherein the processor executable program instructions, when executed by the processor, cause the processor to perform operations comprising:
measure the ultraviolet LED distance from the surface based on receiving sensor data captured by the proximity sensor;
determine, based on the ultraviolet LED distance from the surface, ultraviolet LED illumination parameters effective to disinfect a surface portion exposed to the light emitted by the ultraviolet LED when the ultraviolet LED is illuminated, wherein the ultraviolet LED illumination parameters further comprise:
ultraviolet LED illumination time determined as a function of the area of the surface portion exposed to the light; and,
ultraviolet LED illumination energy determined as functions of the area of the surface portion illuminated by the ultraviolet LED and the LED distance from the surface; and,
automatically disinfect the surface portion by governing the ultraviolet LED illumination parameters adjusted as a function of the area of the surface portion illuminated by the ultraviolet LED and apparatus motion determined based on accelerometer data;
determine if the area of the surface portion exposed to the light has been disinfected, based on the ultraviolet LED illumination time, the ultraviolet LED illumination energy, and the area of the surface portion exposed to the light; and,
in response to determining the area of the surface portion exposed to the light has been disinfected:
provide a user detectable indication the surface portion has been disinfected; and,
direct the user to move the apparatus to an adjacent surface portion overlapping the disinfected surface portion.

21. The apparatus of claim 20, wherein the apparatus further comprises a flexible printed circuit strip operably retaining the processor, accelerometer, ultraviolet LED, passive infrared proximity sensor, and memory, wherein the flexible printed circuit strip has two ends, wherein each flexible printed circuit strip end includes a clasp configured to releasably secure the flexible printed circuit strip to a human wrist based on joining each end to the other end when the flexible printed circuit strip is looped to subsume the wrist, and wherein the ultraviolet LED is configured in the flexible printed circuit strip with a counterbalancing weight adapted to direct the ultraviolet LED down toward a surface.

22. The apparatus of claim 20, wherein the apparatus further comprises a sticker operably retaining the processor, accelerometer, ultraviolet LED, proximity sensor, and memory, wherein the sticker is configured to secure the apparatus to a portable device.
